(19) United States
(12) Patent Application Publication
(10) Pub. No.: US 2018/0338694 A1
(43) Pub. Date: Nov. 29, 2018

(54) METHOD FOR CORRECTING CUFF PRESSURE IN A NON-INVASIVE BLOOD PRESSURE MEASUREMENT

(71) Applicant: Edwards Lifesciences Corporation, Irvine, CA (US)

(72) Inventor: Jacobus Jozef Gerardus Maria Settels, De Hoef (NL)

(21) Appl. No.: 15/984,634

(22) Filed: May 21, 2018

Related U.S. Application Data
(60) Provisional application No. 62/509,877, filed on May 23, 2017.

(51) Int. Cl. A61B 5/0225; A61B 5/022

(52) U.S. Cl. CPC A61B 5/02255; A61B 5/02241

(57) ABSTRACT

A method for correcting cuff pressure in a non-invasive continuous blood pressure measurement with a plethysmograph comprising the steps of: determining a value of the plethysmograph signal corresponding to a predetermined arterial volume, and setting the determined value as set point; comparing measured plethysmograph signals with the set point; monitoring the adjustments of the cuff pressure for a certain amount of heartbeats; storing the pressure adjustments of the cuff during at least one first heartbeat; applying a varying cuff pressure to the pressure cuff during at least one second heartbeat and measuring the corresponding signals of the plethysmograph; using the stored pressure adjustments of the first heartbeat, the cuff pressure and plethysmograph signal of the second heartbeat, to determine a pressure-volume relation; and determining a value of the plethysmograph signal corresponding to predetermined arterial volume, based on the determined relation; and setting the determined value as new set point.

### CROSS-REFERENCE TO RELATED APPLICATIONS

This application claims priority to U.S. Provisional Patent Application No. 62/509,877, filed May 23, 2017, the entire contents of which are incorporated herein by reference.

### BACKGROUND

The present invention relates to a method for correcting cuff pressure in a non-invasive continuous pressure blood pressure measurement, as well as to a device for carrying out said method.

It has been known for several years to measure blood pressures wherein a pressure cuff is placed around a body extremity, such as a finger. EP 0 048 060 for instance describes that the pressure of a fluid inside the pressure cuff is controlled on the basis on a signal of an optical plethysmograph by a pressure valve, in turn controlled by a control loop.

The signal of the optical plethysmograph is representing the volume of blood inside the blood vessels of the finger. The more blood inside the vessels, the more light from a light source of the plethysmograph is absorbed, which results in a lower signal of the detector side of the plethysmograph (and vice versa). During every heartbeat, blood is forced through the blood vessels in the finger, causing the vessels to expand and allow more blood to flow through the vessels. This also causes a volume increase of the vessels, and thus a signal decrease of the plethysmograph.

In the known method, the cuff pressure of the pressure cuff is controlled such that the signal of the plethysmograph, and thus the volume of blood inside the blood vessels, is kept constant. The pressure exerted on the internal blood vessel walls is continuously counteracted by a pressure exerted by the pressure cuff on the external blood vessel walls, which results in a constant diameter of the blood vessels and an unloading of the vessels. The counter pressure exerted by the pressure cuff is a measure for the actual blood pressure inside the blood vessel, and allows for a continuous non-invasive blood pressure measurement.

This control is arranged such that at any moment the difference between a servo reference level or setpoint value for the diameter of the blood vessels and the actual plethysmograph signal or real value is minimized, ideally to zero. The servo-reference level in the known method is initially determined automatically and the servo feedback control is operated in a way such that the cuff pressure continuously corresponds substantially with the momentary arterial pressure under the cuff both for pulsations and for absolute pressure level.

The known method requires correction of the reference or set point value over time. This correction is required mainly due to changes in the physiological status of the measured body part. U.S. Pat. No. 4,510,940 for instance describes a method and a device for correcting the cuff pressure in the indirect, non-invasive and continuous measurement of the blood pressure in a part of the body by using a plethysmograph in a fluid-filled pressure cuff, an electronic control circuit, and an electric pressure valve. The cuff pressure is controlled by the plethysmograph signal in closed-loop operation with the aid of a servo-reference level obtained via a memory circuit. The servo-reference level, in operation of the device, is adjusted by opening the closed loop of the control circuit for a short interval, after which, in open-loop operation the cuff pressure is adjusted at an intermediate pressure derived from the pressure last measured and the servo-reference level is adjusted via the memory circuit.

Although these methods work, the servo-reference level is adjusted or corrected based only on volume measurements of the plethysmograph as a function of time, over only a part of the heart beat duration, and with only constant cuff pressures. These reference computations make assumptions on the blood pressure levels inside the vessels over the time of the correction computation and are sensitive to external influences. Also, these methods are relatively slow. For adjustment determination of the reference level, often several heartbeats are required to determine the response in volume to many different pressures in the pressure cuff, during which heartbeats a measurement of the blood pressure cannot be performed.

It is therefore an objective of the present invention to provide an improved method for correcting cuff pressure in a non-invasive continuous blood pressure measurement.

The invention thereto provides a method for correcting cuff pressure in a non-invasive blood pressure measurement with a plethysmograph in a fluid filled pressure cuff wrapped around a body part, such as a finger, comprising the steps of: varying pressures inside the pressure cuff and measuring the corresponding signals of the plethysmograph; determining a value of the plethysmograph signal corresponding to a predetermined arterial volume, in particular the un-stretched or unloaded arterial volume; and setting the determined value as set point; comparing measured plethysmograph signals with the set point for a servo system; adjusting cuff pressure in the pressure cuff to minimise the difference between the measured signals and the set point; monitoring the cuff pressure for a certain amount of heartbeats; storing the pressure adjustments of the cuff during at least one first heartbeat; applying a varying cuff pressure to the pressure cuff during at least one second heartbeat and measuring the corresponding signals of the plethysmograph; using the stored pressure adjustments of the at least one first heartbeat, and the cuff pressure and plethysmograph signal of the at least one second heartbeat, to determine a pressure-volume relation of the blood vessel under the cuff; and determining a value of the plethysmograph signal corresponding to predetermined arterial volume, preferably an un-stretched arterial volume, based on the determined relation; and setting the determined value as new set point. A fluid according to the invention may for instance be a liquid such as water, or a gas like air.

In the method according to the invention, the blood pressure inside the blood vessel is followed for a certain period of time, to monitor the blood pressure. To do so, a first set point is determined, which approximately corresponds to an unloaded volume of the blood vessel. The pressure in the cuff is adjusted to keep the volume constant, and this pressure is thus a measure for the internal blood pressure (or intra-arterial pressure). Due to multiple factors, this set points typically drifts with time such that calibration, or setting a new set point, is required after a certain period of time.

The set point, or unloaded volume of the blood vessel, can be determined based on a pressure-volume relation between the transmural pressure, which is the internal blood pressure (or intra-arterial pressure) minus the cuff pressure, and the volume of blood inside the blood vessel. The volume is determined by the plethysmograph signal. In the determination of the transmural pressure, the cuff pressure is available and adjustable, but the present (or actual) internal blood pressure is an unknown.

However, changes between blood pressure waveforms generally occur gradually, and if they differ from one heartbeat to the next, the changes are mostly in pulse pressure, which changes can be predicted based on changes of the duration of the heart beat and compensated for. In the determination of the pressure-volume relation according to the invention, the second set of blood pressure waveform for the second heart beat is assumed to be identical to the first set of blood pressure waveform or assumed to be correctly adapted from the first based on heart beat interval. Instead of the present blood pressure in the blood vessel, the blood pressure of the first, preceding set is used. The pressure of the cuff can now be chosen dynamically to cover a range of pressures suitable for the determination of the pressure-volume relation.

The advantage of such method is that a large dynamic range of transmural pressures may be used to compute an actual pressure-volume relation of the blood vessel on which the set point is based, whereas in the prior art the set point is based only a determination on volume information over time.

Another advantage of such method is that it is relatively fast. Because the internal blood pressure, or intra-arterial pressure, is assumed to be known, only one or two heartbeats are required which can be used to dynamically cover a large range of transmural pressures, as the cuff pressure may be varied based on the information from the assumed intra-arterial pressure.

Another advantage of such method is that it allows the analysis of visco-elastic properties of the arterial wall of the blood vessel by determining the phase relationship, compliance and hysteresis characteristics between the transmural pressure dynamics and the resulting volume dynamics

The beat duration of the second heart beat can be determined from the first heartbeat, and the pulse pressure of the second heart beat can be adapted when the beat duration differs from the first heartbeat.

The at least one first heartbeat and the at least one second heartbeat may comprise the same number of heartbeats, for instance one or two heartbeats.

The method according to the invention may repeat the monitoring of blood pressure and correcting the set point after a number of heartbeats. Because the correction of the set point only takes one or a few heartbeats to perform, and the blood pressures during these heartbeat(s) are available as predicted by simulation, only a little bit of actual blood pressure measurement is lost. This allows for as many corrections over time as needed, which improves the quality of the blood pressure determination.

The varying cuff pressure applied during the at least one second heartbeat may comprise a pressure below venous pressure in the body part under the cuff and distal to it. In the finger, the use of a pressure cuff to determine arterial blood pressure in the described way typically blocks or impedes to a major extent the venous return of blood, since the cuff pressure, equalling the arterial pressure, exceeds the venous pressures. The venous blood vessels under the cuff are thus typically collapsed during blood pressure measurements with pressure cuffs. Same is true for the capillary bed under the cuff. This will, after a period of time, result in venous congestion in the body part distal to the cuff, increased venous and capillary pressure and potentially reduced oxygenation in the extremity of the body part, such as the fingertip. When the varying cuff pressure applied during the at least one second heartbeat comprises a pressure below the venous pressure in the fingertip, it allows venous return from the distal part of the extremity to the collecting veins, and avoids to abovementioned congestion problems. In order to achieve this, the cuff pressure below fingertip venous pressure may lie between 0 and diastolic arterial pressure, preferably between 0 and 50 mmHg, in particular around 30 mmHg

The varying cuff pressure applied during the at least one second heartbeat may be a dynamic pressure waveform, such as a sinusoidal waveform. Because the arterial pressure is assumed to be known during the second heartbeat, the pressure-volume relation may be determined with any cuff pressure. The pressure-volume relation relies on the transmural pressure over the arterial wall and the corresponding volume information based on the plethysmograph signal. The transmural pressure is the difference between the cuff pressure and the arterial pressure. As the blood pressure has a somewhat sinusoidal progression itself also, a sinusoidal cuff pressure waveform in counter phase with the blood pressure waveform would allow to generate transmural pressures over a relative large range along with its volume information from the plethysmograph, both positive and negative transmural pressures.

The varying cuff pressure applied during the at least one second heartbeat may also be a ramp waveform, wherein preferably the lowest pressure below diastolic pressure in the body part allows venous return in the body part, and/or the highest pressure is above systolic pressure in the body part. The ramp pressure can be in phase or in counter phase with the blood pressure dynamics, or any phase relationship in between. A ramp in pressure is relatively easy to apply. The ramp waveform may comprise multiple ramps, such that the waveform is more like a saw tooth waveform.

The varying cuff pressure applied during the at least one second heartbeat may be chosen such that the transmural pressure between the blood vessel of the body part and the cuff pressure is set over a predetermined range. The transmural pressure depends on the arterial pressure and the cuff pressure, of which the arterial pressure is known, and the cuff pressure can be set. In order to determine the transmural pressure over a predetermined range, the cuff pressure can be varied based on the arterial pressure. The varying pressure profile may thus be determined based on the stored blood pressure of the at least one first heartbeat.

The method may further comprise the step of determining the central blood pressure based on the measured blood pressure inside the body part, preferably the finger. When the blood pressure inside the body part is transformed in the central blood pressure, measurements of different body parts may be compared, as all can be related to central blood pressure. Central blood pressure is for instance the aortic blood pressure or the brachial blood pressure.

The step of using the stored pressure adjustments of the at least one first heartbeat, and the cuff pressure and plethysmograph signal of the at least one second heartbeat, to determine a pressure-volume relation for instance involves plotting the transmural pressure against the volume signal of the plethysmograph. In each heartbeat, this pressure/volume relation substantially forms a loop, with an upswing and a downswing distinguished from each other because of the hysteresis of the arterial wall. The gradient, or angle/steepness of these swings is a measure for the compliance of the blood vessel being measured. In order to better study this gradient or steepness, instead of the volume one could take the first derivative of the volume signal, and plot this derivative over the transmural pressure. The location where this first derivative is maximal (or where a second derivative would be zero) represents the transmural pressure with maximal compliance. This transmural pressure could be used in the pressure-volume relation to determine the volume where compliance is maximal, and it is this volume which could serve as new set point according to the invention.

The invention further relates to a device for correcting cuff pressure in a non-invasive blood pressure measurement with a plethysmograph in a fluid filled pressure cuff wrapped around a body part, such as a finger, comprising a pressure cuff, provided with: a bladder, for wrapping around the body part and applying counter pressure; a light source, for sending light through the body part, and a light detector, for detecting the light passed through the body part and for providing a signal in dependence of the amount of detected light; a pressure generator, for supplying pressurized fluid to the bladder; a controller, for adjusting the pressure of the fluid supplied to the bladder based on the signal, and for determining the blood pressure inside the body part; a memory, for storing the determined blood pressure of at least one first heartbeat; wherein the controller is arranged to apply a varying dynamic cuff pressure during at least one second heartbeat, and to receive the signal during the at least one second heartbeat, and wherein the controller is arranged to determine a pressure volume relation based on the stored blood pressure of the at least one first heartbeat, the signal of the at least one second heartbeat and the varying cuff pressure applied during the at least one second heartbeat.

The device may further comprise a pressure sensor arranged to determine the pressure inside the bladder. The pressure inside the bladder is an indication of the blood pressure inside the body part when the pressure is adjusted to maintain a constant plethysmograph signal.

The invention will be explained by means of the non-limiting working examples depicted in the following figures. Specifically:

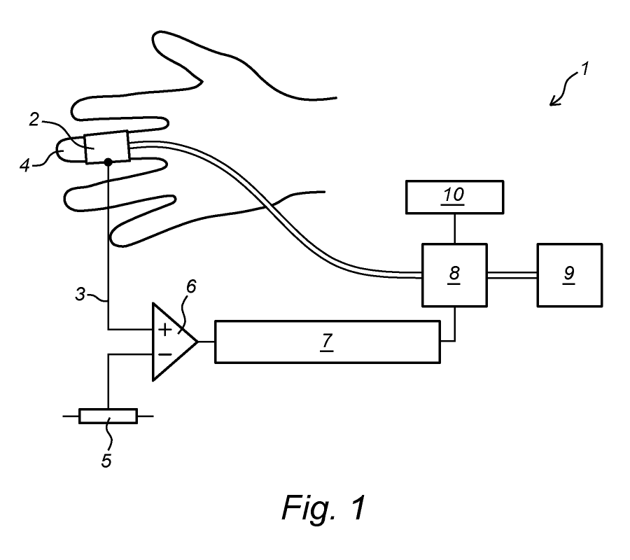

FIG. 1 schematically shows a device for non-invasive blood pressure measurements according to the present invention;

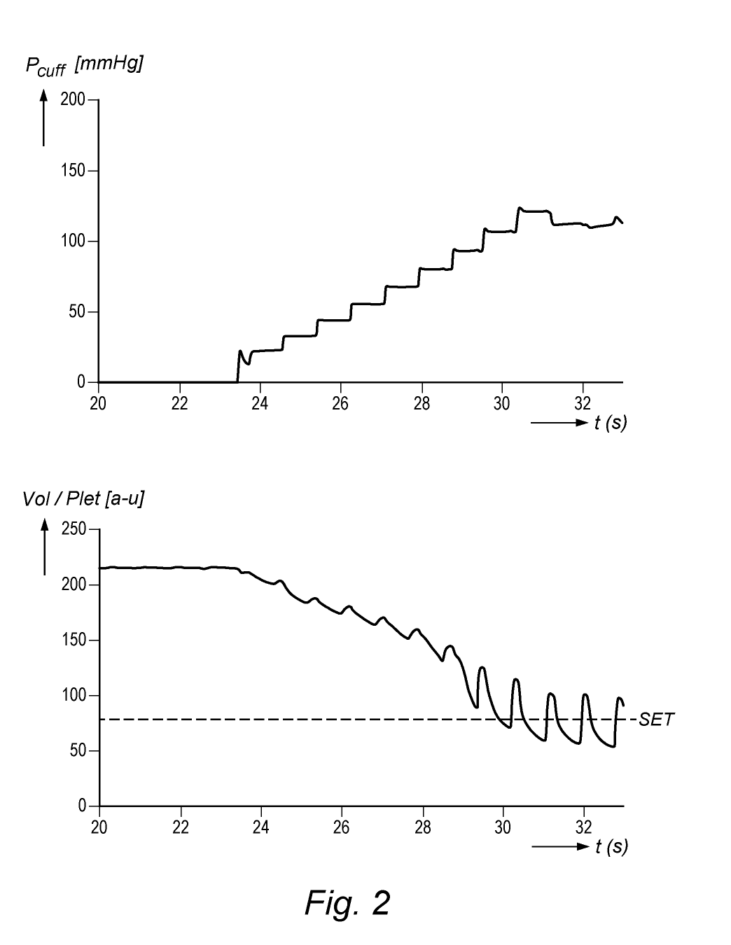

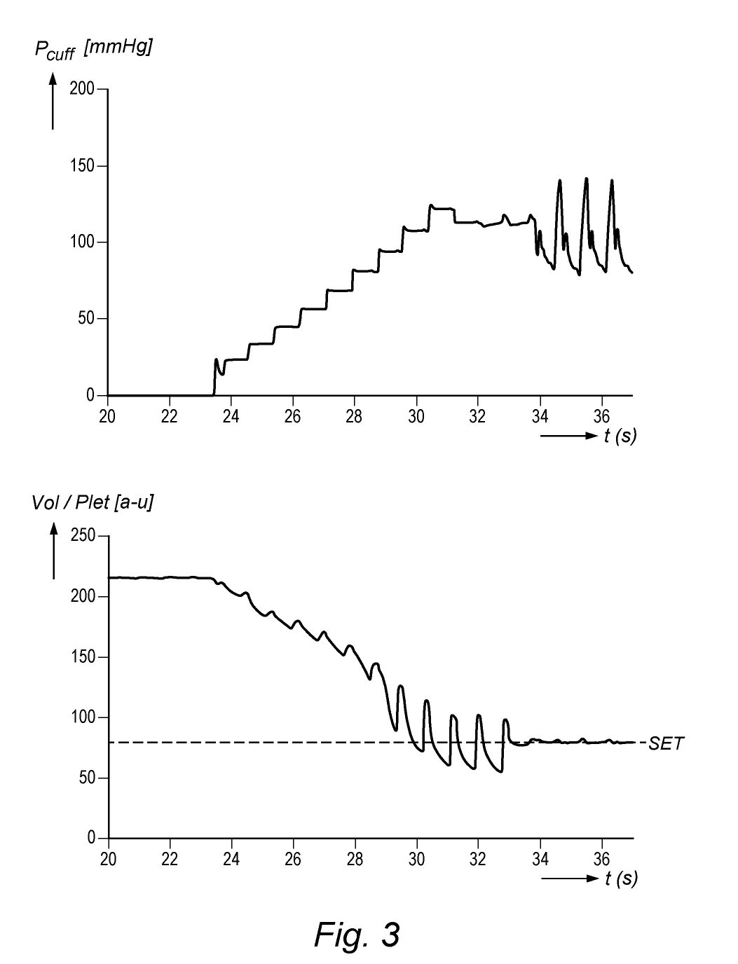

FIG. 2 schematically shows a first approximate determination of the set point according to the present invention;

FIG. 3 schematically shows the monitoring of blood pressure after the first setting of the set point according to the present invention;

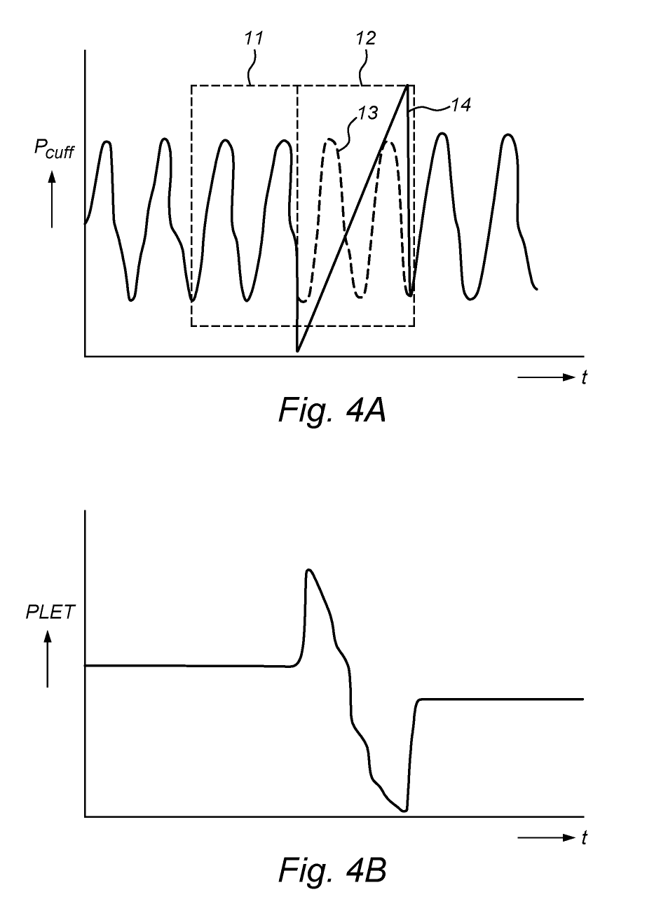

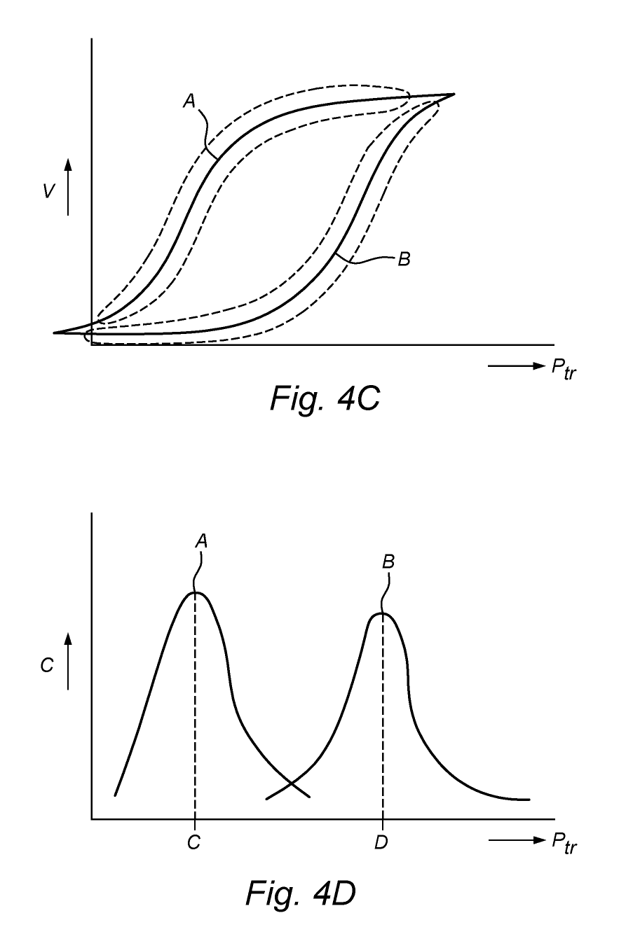

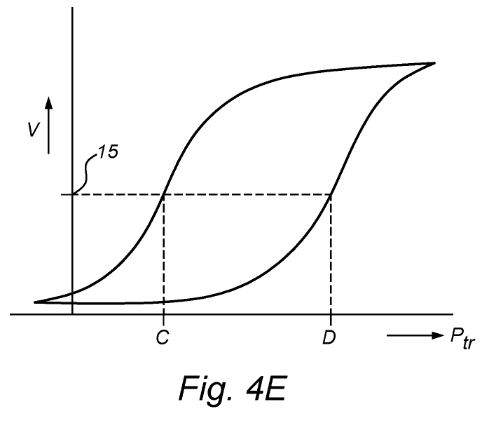

FIGS. 4A-4E schematically shows the determination of a new, changed, set point according to the present invention; and

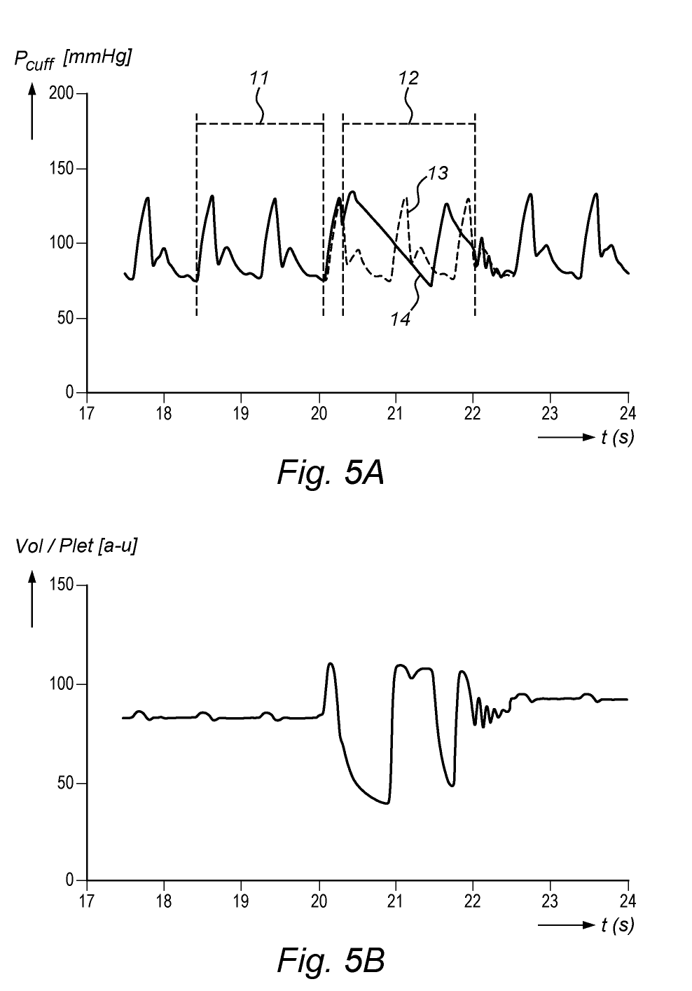

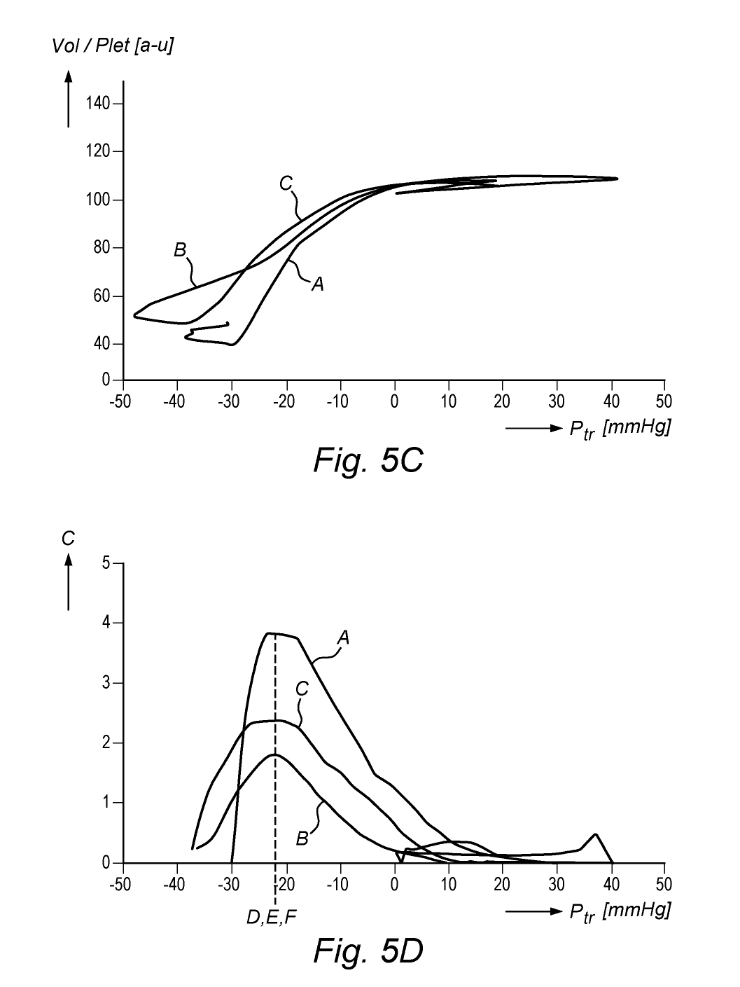

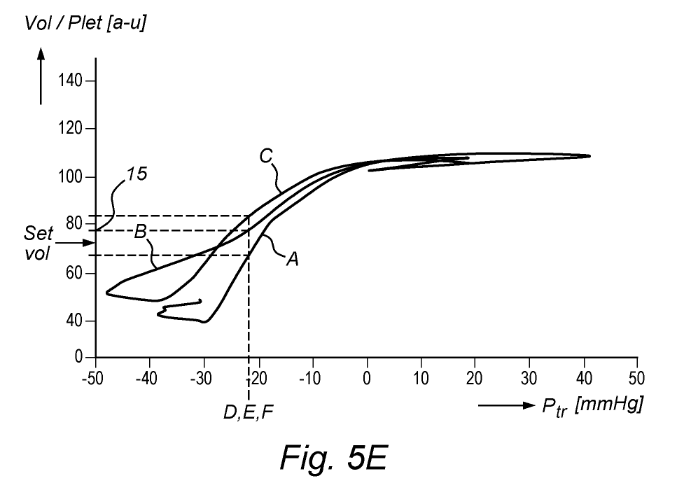

FIG. 5A-5E shows the actual determination of a new, changed, set point according to the present invention using a ramp as the varying cuff pressure and

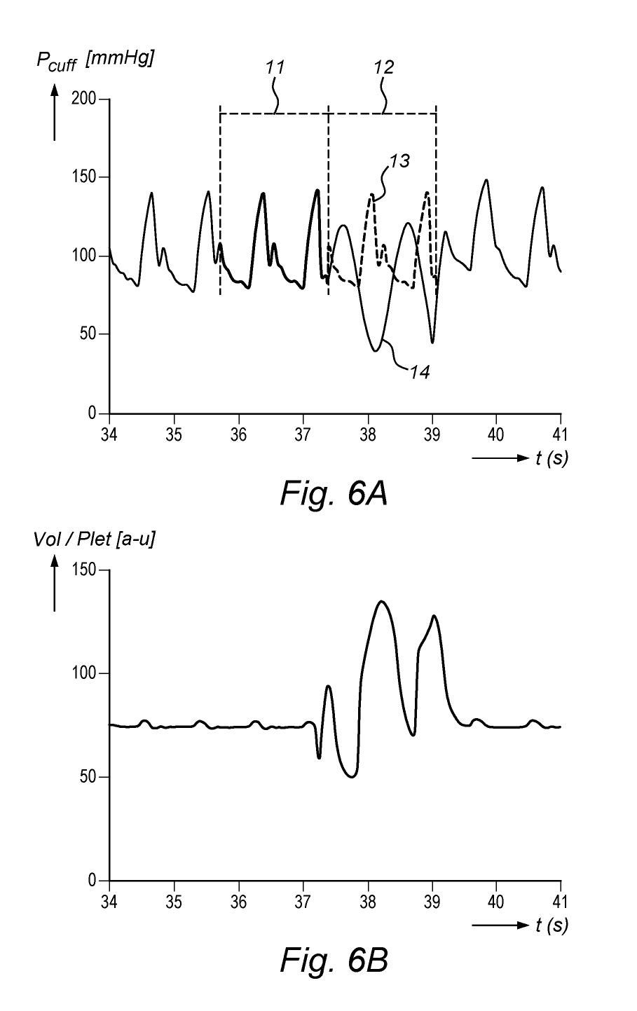

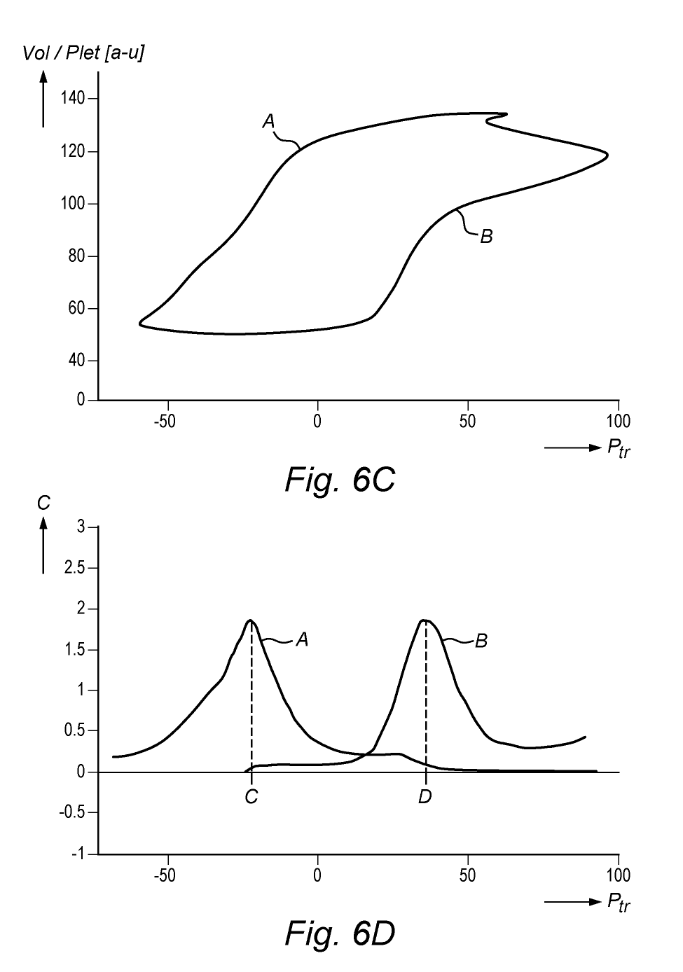

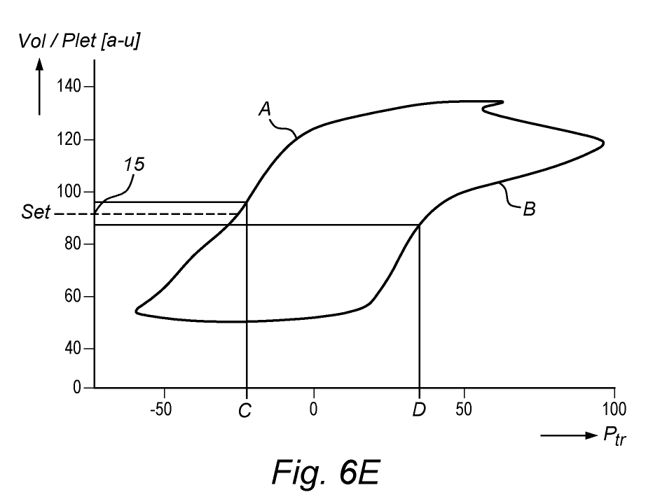

FIG. 6A-6E shows the actual determination of a new, changed, set point according to the present invention using a sinusoidal waveform as the varying cuff pressure.

FIG. 1 schematically shows a device (1) for non-invasive blood pressure measurements, comprising a pressure cuff (2), which generates a signal (3) based on the detected light. This signal (3), representative for the volume of blood in the finger arteries (4) is compared to a set-point (5) by a comparator (6), which comparison is then communicated to a controller (7). Based on the information, the controller (7) in turn controls a control valve (8). The valve (8) regulates the pressure supplied to the pressure cuff (2) by a pump (9). The pressure supplied to the pressure cuff (2) is measured by a transducer (10).

FIG. 2 schematically shows a first determination of the first set point (SET). In the upper graph, the pressures (P_{cuff}) inside the pressure cuff are plotted over time (t). The pressures are varied in this example by applying them in a step pattern with increasing cuff pressures. Such pattern can however be selected from a number of patterns, also for instance including a ramp pattern.

In the bottom graph, the corresponding plethysmograph signal is shown. At low cuff pressures (on the left of the graph), the arteries of the finger are open and a large amount of blood is present in the arteries. The plethysmograph signal (PLET), corresponding to the amount of blood in the measured arteries, is thus relatively high. At high cuff pressures (on the middle/right of the graph), the cuff exerts a large pressure on the finger and the arteries, causing them to collapse at least partially during the diastolic phase of the heartbeat. This collapse of the arteries reduces the blood volume, and thus results in a decreased plethysmograph signal.

The blood pressure in the arteries also pulsates with every heartbeat. Every heartbeat blood is expelled from the heart into the aorta during systole, which is followed by the filling of the heart during diastole. The pressure in the arteries follows the same pattern, and increases due to the expulsion of blood, and decreases during filling of the heart. This pulsation causes an expansion of the arteries in the finger, and thus in a varying volume of blood in the arteries. This varying volume also results in a varying plethysmograph signal.

The variations in the plethysmograph signal are small when the cuff pressure is high (for instance above systolic pressure), as the arteries are subjected to high external pressures, working against the internal pressure variations. The variations in the plethysmograph signal are also small when the cuff pressure is low (for instance below diastolic pressure), as then arteries are loaded by the blood pressure from within.

When the external pressure of the pressure cuff and the internal blood pressure of the arteries are close or equal, they balance each other out. This is also referred to as the unloaded state of the arteries, wherein the pressure difference over the arterial walls (also referred to as transmural pressure) is zero. In this state, the varying blood pressure causes, relatively unobstructed and directly, the expansion of the arteries. It is in this state that the variations in the plethysmograph signal are largest. The initial set point, is for instance chosen as the middle between the top and bottom of the plethysmograph signal where the variations are largest. This procedure does not provide a very accurate volume set point, but it suffices to start a measurement with the new set point adjustments as described, which quickly converges to a stable and more accurate volume set point value.

FIG. 3 schematically shows the monitoring of blood pressure after the setting of the initial set point. The pressure in the cuff (P_{cuff}) is varied such that the plethysmograph signal (PLET) is kept constant, at the set point value. The pressure in the cuff is now representing the blood pressure inside the arteries.

The set point, representing the predetermined or unloaded volume of the arteries, changes over time. Performing the same step pattern in cuff pressures to determine the changed set point takes a relative long time to do, and is not very accurate and the blood pressure measurement is then temporarily not available.

FIG. 4 schematically shows the determination of the information leading to a new, changed, set point. FIG. 4A shows a changing pressure in the cuff, P_{cuff}, and FIG. 4B shows the changing plethysmograph signal over that time, PLET. In FIG. 4A, the blood pressure is monitored for a number, for example four, heartbeats before determination of the new set points. During this monitoring the plethysmograph signal (PLET) in FIG. 4B is kept constant at or around the previously determined set point, and the pressure in the cuff is adjusted accordingly.

The cuff pressures, representing blood pressures, of the last two heartbeats (11) are stored. In FIG. 4 two heartbeats are stored, but the same can hold for one or more heartbeats. For the next two heartbeats (12), the blood pressures are assumed to be identical to the stored heartbeats (11), as indicated with the dotted line (13) in the upper graph of FIG. 3. During these two heartbeats (12), a varying cuff pressure pattern (P_{cuff}) is applied to the pressure cuff, as indicated by e.g. the ramp (14). The plethysmograph signal (PLET) is measured over this range of cuff pressures.

The blood pressures during the two heartbeats are known, as they are assumed to be the same as the previous two heartbeats. In addition, they can be slightly modified based on the variation in heart beat interval. The cuff pressures (P_{cuff}) during the two heartbeats are the pressures according to the ramp (14), and the blood volume in the arteries is provided by the plethysmograph signal (PLET). The pressure over the arterial walls, or transmural pressure, is the difference between the cuff pressure and the arterial blood pressure, and can thus be determined. The volume can subsequently be plotted as a function of transmural pressure.

FIG. 4C schematically shows a pressure volume relation according to the invention, which can be obtained using the steps described with regard to FIGS. 4A and 4B. For sake of simplicity, the relation is shown for a single heartbeat. On the horizontal axis, the transmural pressure (Ptr) is shown, which pressure is the internal blood pressure, or arterial pressure, minus the cuff pressure. On the vertical axis, a volume (in internal units) is shown, which is provided by the signal received by the plethysmograph. In the shown relation, two curves (A, B) can be identified, curve A being related to the inflow and curve B being related to the outflow of blood. The gradient, or steepness, of each of the curves is a measure for the compliance of the blood vessel, and is the derivative of these curves.

FIG. 4D schematically shows the (first) derivative of these curves (A, B) schematically. For each of these curves (A, B) a maximum can be determined, which corresponds to a certain transmural pressure (Ptr), one for curve A (C), and one for curve B (D). FIG. 4E schematically shows how these transmural pressures where compliance is maximal can be used to determine a new set point. The new (volume) set point is the volume (15) in the pressure-volume relation as for instance shown in FIG. 4C which corresponds to the transmural pressures where compliance is maximal. This volume (15), will be the new (volume) set point (Vₛₑₜ) of the plethysmograph. It should be noted that the actual transmural pressure is used in this method (just) for referencing, and its precise value is of minor relevance. When the two curves (A, B) would have volumes (15) which are not coinciding, but would be different, the new set point could for instance be the average volume of the two volumes (15).

FIGS. 5A-5E show the determination of a new, changed, set point according to FIG. 4 in an actual measurement, using a ramp as the varying cuff pressure. The steps of the determination are according to the ones of FIG. 4.

FIGS. 6A-6E show the determination of a new, changed, set point in an actual measurement, using a sinusoidal waveform as the varying cuff pressure. The steps of the determination are according to the ones of FIGS. 4 and 5.

By including cuff pressure profiles that contain both transitions from low pressure, e.g. below diastolic to high pressure, e.g. above systolic and transitions from high to low pressure, the phase relationship between pressure and volume will show in the pressure-volume diagram as hysteresis if that is present, and provide information on the arteries. The hysteresis will provide at least two different values for the unloaded volume at zero transmural pressure. These values can be used to compute a more accurate determination of the set point at the unloaded volume, by computing an average of the two or more values, where the average can be a weighted average.

Because of its visco-elastic properties, the arterial wall of the body part under the cuffs has low pass filtering characteristics which can be determined by applying a step function in transmural pressure up and down around a predetermined cuff pressure level; this level is preferably at mean arterial pressure or at mid pressure, half way in between systolic and diastolic pressure. The varying cuff pressure in this case has the profile of a step up or down, followed by cuff pressure tracking the pressure inside the finger artery over a certain period, thus keeping the transmural pressure constant over a certain period which preferably is in the order of several 100 milliseconds. The resulting volume transient will have a characteristic which can be approximated by a first order time constant and estimated by the controller. This time constant will vary from person to person, and will vary over time within a person. For better robustness, the average value of the time constant up and the time constant down can be determined. The resulting time constant value can be used to individualize an important compensation component in the servo feedback loop that maintains the volume to the set point value; to individualize to each patient, and to adapt to changes over time. As a result, the servo will have improved bandwidth which will result in higher quality waveform details. The time constant can also be determined using a step in cuff pressure up or down, followed by a constant cuff pressure over a certain period. In this case, however, the transmural pressure will not be kept constant and the resulting time constant estimate will have a bias depending on the individual pressure shape in the finger artery.

It will be apparent that the invention is not limited to the exemplary embodiments shown and described here, but that within the scope of the appended claims numerous variants are possible which will be self-evident to the skilled person in this field.

What is claimed is:

1. A method for correcting cuff pressure in a non-invasive continuous blood pressure measurement with a plethysmograph in a fluid filled pressure cuff wrapped around a body part, the method comprising the steps of:
a) varying pressures inside the pressure cuff and measuring the corresponding signals of the plethysmograph;
b) determining a value of the plethysmograph signal corresponding to an unloaded arterial volume; and setting the determined value as an initial set point;
c) comparing measured plethysmograph signals with the set point;
d) adjusting cuff pressure in the pressure cuff to minimise the difference between the measured signals and the set point;
e) monitoring the cuff pressure for a number of heartbeats;
f) storing the pressure adjustments of the cuff during at least one first heartbeat;
g) applying a varying cuff pressure to the pressure cuff during at least one second heartbeat and measuring the corresponding signals of the plethysmograph
h) using the stored pressure adjustments of the at least one first heartbeat, and the cuff pressure and plethysmograph signal of the at least one second heartbeat, to determine a pressure-volume relation
i) determining a value of the plethysmograph signal corresponding to an un-stretched arterial volume, based on the determined relation; and setting the determined value as a new set point.

2. A method according to claim 1, comprising step j) of repeating steps c)-i).

3. A method according to claim 1, wherein the varying cuff pressure applied in step g) comprises a pressure below venous pressure in the body part distal to the cuff.

4. A method according to claim 3, wherein the pressure below venous pressure in the body part distal to the cuff reaches values below diastolic blood pressure, preferably between 0 and 50 mmHg, in particular around 30 mmHg

5. A method according to claim 1, wherein the varying cuff pressure applied in step g) is a dynamic pressure waveform, in which the lowest pressure is below diastolic pressure in the body part, which allows venous return in the body part, and the highest pressure is above systolic pressure in the body part.

6. A method according to claim 1, wherein the varying cuff pressure applied in step g) is a ramp waveform in which the lowest pressure is below diastolic pressure in the body part, which allows venous return in the body part, and the highest pressure is above systolic pressure in the body part.

7. A method according to claim 1, wherein the varying cuff pressure applied in step g) comprises a transition from a low pressure below diastolic pressure to a high pressure above systolic pressure, and thereafter multiple transitions between said low and high pressures.

8. A method according to claim 1, and further comprising a step of applying a cuff pressure having a step function around a predetermined cuff pressure level, which level is one of (i) mean arterial pressure and (ii) a pressure halfway between systolic and diastolic pressure.

9. A method according to claim 1, wherein the pressure-volume relation is determined by the signal of the plethysmograph and the transmural pressure between the blood vessel of the body part and the cuff pressure.

10. Method according to claim 1, wherein the varying cuff pressure applied during step g) is chosen such that the transmural pressure between the blood vessel of the body part and the cuff pressure is set over a predetermined range.

11. Method according to claim 1, wherein the varying cuff pressure applied during step g) is determined based on stored blood pressure of the at least one first heartbeat.

12. Method according to claim 1, wherein in step b), the set point value is the volume wherein the amplitude of the plethysmograph signal is maximal.

13. Method according to claim 1, and further comprising a step of determining the central blood pressure based on the measured blood pressure inside the body part, preferably the finger.

14. Method according to claim 1, wherein in step i), the new set point is determined in relation to the volume where a compliance of the measured body part, such as the finger artery, is maximal.

15. Device for correcting cuff pressure in a non-invasive blood pressure measurement with a plethysmograph in a fluid filled pressure cuff wrapped around a body part, such as a finger, comprising:
a) a pressure cuff, provided with:
a. a bladder, for wrapping around the body part and for applying a counter pressure;
b. a light source, for sending light through the body part, and
c. a light detector, for detecting the light passed through the body part and for providing a signal in dependence of the amount of detected light;
b) a pressure generator, for supplying pressurized fluid to the bladder;
c) a controller, for adjusting the pressure of the fluid supplied to the bladder based on the signal, and for determining the blood pressure inside the body part;
d) a memory, for storing the determined blood pressure of at least one first heartbeat;
e) wherein the controller is arranged to apply a varying cuff pressure during at least one second heartbeat, and to receive the signal during the at least one second heartbeat, and
f) wherein the controller is arranged to determine a pressure volume relation based on the stored blood pressure of the at least one first heartbeat, the signal of the at least one second heartbeat and the varying cuff pressure applied during the at least one second heartbeat.
